(19) United States
(12) Patent Application Publication
(10) Pub. No.: US 2024/0156705 A1
(43) Pub. Date: May 16, 2024

(54) COSMETIC COMPOSITION COMPRISING AT LEAST ONE POLAR OIL, A POLYOL AND AT LEAST ONE HYDROPHILIC ACTIVE AGENT

(71) Applicant: L'OREAL, Paris (FR)

(72) Inventors: Sabine VRIGNAUD BARRETEAU, Chevilly la Rue (FR); Pamella WANG, Chevilly la Rue (FR)

(73) Assignee: L'OREAL, Paris (FR)

(21) Appl. No.: 18/570,450

(22) PCT Filed: Jun. 21, 2022
(86) PCT No.: PCT/EP2022/066869

§ 371 Date: Dec. 14, 2023

(30) Foreign Application Priority Data
Jun. 21, 2021 (FR) FR2106548

(51) Int. Cl. A61K 8/49; A61K 8/34; A61K 8/36; A61K 8/37; A61Q 19/00

(52) U.S. Cl. CPC A61K 8/498; A61K 8/342; A61K 8/345; A61K 8/361; A61K 8/37; A61Q 19/00; A61K 2800/10; A61K 2800/262

(57) ABSTRACT

The present invention relates to a transparent single-phase cosmetic composition comprising:
from 0.1% to 89% by weight of at least one polar oil with respect to the total weight of the composition, said polar oil comprising at least one compound chosen from isopropyl myristate, di-alkyl carbonates, the 2 alkyl chains optionally being identical or different, and mixtures thereof;
at least 10% by weight with respect to the total weight of the composition of water-miscible organic solvent(s) chosen from polyols having 2 to 20 carbon atoms optionally in a mixture with at least one C₂-C₆ aliphatic monoalcohol; and
at least one hydrophilic active agent,
   said composition comprising less than 8% by weight of water with respect to the total weight of the composition.

The present invention relates to a transparent single-phase cosmetic composition, comprising:
from 0.1% to 89% by weight of at least one polar oil with respect to the total weight of the composition, said polar oil comprising at least one compound chosen from isopropyl myristate, di-alkyl carbonates, the 2 alkyl chains optionally being identical or different, octyldodecanol, and mixtures thereof;
at least 10% by weight with respect to the total weight of the composition of water-miscible organic solvent(s) chosen from polyols having 2 to 20 carbon atoms optionally in a mixture with at least one C₂-C₆ aliphatic monoalcohol; and
at least one hydrophilic active agent,
   said composition comprising less than 8% by weight of water with respect to the total weight of the composition.

The present invention relates in particular to a transparent single-phase cosmetic composition, comprising:
from 0.1% to 89% by weight of at least one polar oil with respect to the total weight of the composition, said polar oil comprising at least one compound chosen from isopropyl myristate, di-alkyl carbonates, the 2 alkyl chains optionally being identical or different, and mixtures thereof;
at least 10% by weight with respect to the total weight of the composition of water-miscible organic solvent(s) chosen from polyols having 2 to 20 carbon atoms optionally in a mixture with at least one C₂-C₆ aliphatic monoalcohol; and
at least one hydrophilic active agent,
   said composition comprising less than 8% by weight of water with respect to the total weight of the composition.
   The present invention also relates to the cosmetic use of such a composition, as well as a non-therapeutic cosmetic keratin material care method using such a composition.

In the cosmetic field of care, cosmetic compositions making it possible to obtain both good sensory properties as well as a care effect are increasingly sought by users. Care effect denotes for example an effect countering the drying or aging of keratin materials and particularly the skin, this care effect being provided by active agents. As such, hydrophilic active agents, for example of C-glycoside derivative type, prove to be particularly of interest; in particular, these compounds are particularly effective for treating skin aging problems.

Furthermore, oily dosage forms are of interest for enhancing the delivery of an active agent for application on keratin materials, preferably the skin, and thus the efficacy thereof, in particular the efficacy thereof for treating skin aging problems. Oily dosage forms are also increasingly sought in the cosmetic field and more specifically in the field of skin care, particularly as they provide nourishment after application.

However, obtaining this dual effect (good sensory properties and care effect) requires, on one hand, being able to introduce one (or more) hydrophilic active agent(s) in sufficient quantity in the composition, and, on the other, overcoming the drawbacks associated with oily dosage forms. Yet, the hydrophilic nature of said active agent makes it difficult to incorporate this type of compound in oily dosage forms, which generally comprise little water.

Consequently, the need remains for single-phase cosmetic compositions rich in oil(s), and comprising an effective quantity of hydrophilic active agent(s), which help enhance the delivery of these active agents, which are stable, and which have good sensory properties, particularly which have a reduced absorption time. It also remains the need for a single-phase cosmetic composition having a less oily finish.

The Applicant surprisingly observed that an oily cosmetic composition comprising at least one specific polar oil, at least one hydrophilic active agent, and a C₂-C₆ aliphatic monoalcohol and/or polyols at defined contents, makes it possible to reduce the absorption time thereof after application, while being stable. This composition also makes possible to reduce the oily finish. This composition is rich in oily phase and transparent single-phase.

Thus, the present invention relates to a transparent single-phase cosmetic composition comprising:
from 0.1% to 89% by weight of at least one polar oil with respect to the total weight of the composition, said polar oil comprising at least one compound chosen from isopropyl myristate, di-alkyl carbonates, the 2 alkyl chains optionally being identical or different, octyldodecanol, and mixtures thereof;
at least 10% by weight with respect to the total weight of the composition of water-miscible organic solvent(s) chosen from polyols having 2 to 20 carbon atoms optionally in a mixture with at least one C₂-C₆ aliphatic monoalcohol; and
at least one hydrophilic active agent,
   said composition comprising less than 8% by weight of water with respect to the total weight of the composition.

In particular, the present invention relates to a transparent single-phase cosmetic composition comprising:
from 0.1% to 89% by weight of at least one polar oil with respect to the total weight of the composition, said polar oil comprising at least one compound chosen from isopropyl myristate, di-alkyl carbonates, the 2 alkyl chains optionally being identical or different, and mixtures thereof;
at least 10% by weight with respect to the total weight of the composition of water-miscible organic solvent(s) chosen from polyols having 2 to 20 carbon atoms optionally in a mixture with at least one C₂-C₆ aliphatic monoalcohol; and
at least one hydrophilic active agent,
   said composition comprising less than 8% by weight of water with respect to the total weight of the composition.

The composition according to the invention is stable.

A composition is referred to as stable when the macroscopic appearance (clearness and homogeneity) does not change after at least 24 hours. In particular, the composition according to the invention has a clear and homogeneous appearance.

For the purposes of this invention, the term “clear” denotes a composition having a transparent appearance.

For the purposes of this invention, the term “homogeneous” denotes a composition consisting of a single phase, in other words single-phase.

The composition according to the invention is preferably intended for a topical application, particularly on keratin materials, in particular the skin. It particularly comprises a physiologically acceptable medium, i.e., a medium compatible with all keratin materials, in particular the skin.

For the purposes of this invention, the term keratin materials denotes the skin and the appendages thereof.

The term “skin” denotes facial skin and/or body skin, and the scalp.

The term “appendages” denotes eyelashes, eyebrows, nails and hair, and particularly eyelashes and hair.

The present invention also relates to a method of cosmetic treatment of keratin materials, comprising application of a composition according to the invention on said keratin materials. Moreover, the invention also relates to the use of said composition in the cosmetic field, in particular for body or facial skin care.

Preferably, the composition according to the invention comprises less than 5% by weight of surfactant, preferably less than 3% by weight of surfactant, more preferentially less than 2% by weight of surfactant, preferably less than 1% by weight of surfactant with respect to the total weight of the composition. Preferably, the composition is free from surfactant.

For the purposes of this invention, the term “surfactant” denotes an amphiphilic molecule, i.e., it has two parts of different polarity, in general one is lipophilic (soluble dispersible in an oily phase), the other is hydrophilic (soluble or dispersible in water). Surfactants are characterized by the value of the HLB (Hydrophilic Lipophilic Balance) thereof, the HLB being the ratio between the hydrophilic part and the lipophilic part in the molecule. The term HLB is well known to those skilled in the art and is described for example in “The HLB system. A time-saving guide to Emulsifier Selection” (published by ICI Americas Inc; 1984). For emulsifying surfactants, the HLB generally ranges from 3 to 8 for the preparation of W/0 emulsions and from 8 to 18 for the preparation of O/W emulsions. The HLB of the surfactant(s) used according to the invention can be determined using the GRIFFIN method or the DAVIES method.

Preferably, the composition according to the invention does not comprise emulsifying surfactants such as polyoxyethylenated castor oil, fatty acid and sorbitol and/or sorbitan esters optionally oxyethylenated such as polysorbates, phospholipids, polyethylene glycol and fatty acid esters such as PEG-15 stearate, as well as lecithin.

Preferably, the composition according to the invention does not comprise castor oil, also known as Ricinus communis seed oil.

The composition according to the invention comprises less than 8% by weight of water with respect to the total weight of the composition, preferably less than 4% by weight of water with respect to the total weight of the composition, preferably less than 3% by weight of water with respect to the total weight of the composition, preferably less than 2% by weight of water with respect to the total weight of the composition, preferably less than 1.5% by weight of water with respect to the total weight of the composition, preferably less than 1.1% by weight of water with respect to the total weight of the composition.

The composition according to the invention differs in particular from emulsions in that it is not presented in the form of a dispersion of two mutually non-miscible liquids at ambient temperature (20-25° C.). The composition according to the invention is indeed transparent single-phase. It is single-phase as it has no dispersed phase (also known as discontinuous phase) in droplet form in a dispersing phase (also known as continuous phase).

It is also transparent. The term transparent composition denotes according to the present invention a composition with a turbidity value of less than 200 NTU, preferably less than 150 NTU, and preferably less than 100 NTU. Preferably, the turbidity of compositions is equal to at least 1 NTU.

NTUs (nephelometric turbidity units) are units for measurement of the turbidity of a composition. The turbidity measurement is made, for example, with a model 2100P turbidity meter made by the Hach Company, the tubes used for the measurement being references AR397A cat 24347-06. The measurements are made at ambient temperature (from 20° C. to 25° C.).

Preferably, the composition is transparent and has a turbidity value equal to between and 200 NTU, preferably between 1 and 150 NTU, and preferably less than 100 NTU.

The composition according to the invention is therefore neither opalescent, turbid, opaque, nor bi-phase.

The macroscopic analysis (with the naked eye) of the composition according to the invention shows a transparent and single-phase appearance stable over time at ambient temperature, 4° C. and 45° C.

### Polar Oil(s)

The composition according to the invention comprises from 0.1%% to 89% by weight, preferably from 2.5% to 89% by weight, of at least one polar oil with respect to the total weight of the composition, said polar oil comprising at least one compound chosen from isopropyl myristate, di-alkyl carbonates, the 2 alkyl chains optionally being identical or different, octyldodecanol, and mixtures thereof.

The composition according to the invention comprises in particular from 0.1%% to 89% by weight, preferably from 2.5% to 89% by weight, of at least one polar oil with respect to the total weight of the composition, said polar oil comprising at least one compound chosen from isopropyl myristate, dicaprylyl carbonate and mixtures thereof. Preferably, the composition according to the invention comprises a total quantity of polar oil(s) ranging from 35% to 80% by weight, preferably from 40% to 75% by weight, preferably from 50% to 65% by weight with respect to the total weight of the composition.

The term “oil” denotes any fatty substance in liquid form at ambient temperature (25° C.) and at atmospheric pressure. Among the oils which can be used in the present invention, mention can be made of: volatile or non-volatile oils, these oils can be hydrocarbon oils particularly of animal or plant origin, synthetic oils, silicone oils, fluorinated oils, or mixtures thereof.

For the purposes of this invention, the term “silicone oil” denotes an oil comprising at least one silicon atom, and in particular at least one Si—O group.

The term “hydrocarbon oil” denotes an oil containing essentially hydrogen and carbon atoms and optionally oxygen, nitrogen, sulfur and/or phosphorus atoms.

The term “polar oil”, for the purposes of this invention, denotes an oil wherein the solubility parameter δₐ at 25° C. is different to 0 (J/cm³)^{1/2}. In particular, the term “polar oil” denotes an oil wherein the chemical structure is essentially formed from, or consists of, carbon and hydrogen atoms, and comprising at least one highly electronegative heteroatom such as an oxygen, nitrogen, silicon or phosphorus atom.

The definition of HANSEN three-dimensional solubility parameters are described in the article by C. M. HANSEN: “The three-dimensional solubility parameters” J. Paint Technol. 39, 105 (1967).

According to the Hansen space:
δ_{D} characterizes the LONDON dispersion forces derived from the formation of dipoles induced during molecular shocks;
δₚ characterizes the DEBYE interaction forces between permanent dipoles and the KEESOM interaction forces between induced dipoles and permanent dipoles;
δₕ characterizes the specific interaction forces (such as hydrogen, acid/base, donor/acceptor bonds, etc.);
δₐ is determined by the equation:

δa=(δp²+8h²)½ [Math 1]

The parameters δₚ, δₕ, δ_{D} and δₐ are expressed in (MPa)^{1/2}, a unit equivalent to (J/cm 3)^{1/2}. The Hansen solubility parameters are calculated using HSPiP software version V4.1 based on a chemical structure; the method selected in the software is Y-MB or Yamamoto-Molecular Break.
This software is available to download from the official Hansen parameter and HSPiP software website at the address www.hansen-solubility.com.

Preferably, the polar oils used according to the present invention have a δₐ between 4 and 9.1, preferably a δₐ between 4.5 and 9.1, more preferably between 5 and 8.

The polar oil according to the invention comprises in particular at least one compound chosen from isopropyl myristate, di-alkyl carbonates, the 2 alkyl chains optionally being identical or different, octyldodecanol, and mixtures thereof.

The polar oil according to the invention comprises in particular at least one compound chosen from isopropyl myristate, di-alkyl carbonates, the 2 alkyl chains optionally being identical or different, and mixtures thereof.

Preferably, the di-alkyl carbonate, the 2 alkyl chains optionally being identical or different, is dicaprylyl carbonate, It is particularly marketed under the name Cetiol CC® by Cognis.

Preferably, the composition according to the invention comprises at least isopropyl myristate.

Preferably, the composition according to the invention comprises at least dicaprylyl carbonate.

Preferably, the composition according to the invention comprises at least octyldodecanol.

The composition according to the invention can also comprise other non-volatile or volatile polar oils (additional polar oils). Preferably, it comprises non-volatile additional polar oils.

### Non-Volatile Additional Polar Oils

For the purposes of this invention, the term “non-volatile oil” denotes an oil having a vapor pressure less than 0.13 Pa (0.01 mm Hg), at ambient temperature and atmospheric pressure. The non-volatile additional oils can particularly be selected from non-volatile hydrocarbon oils, where applicable fluorinated, and/or silicone oils.

By way of non-volatile hydrocarbon additional polar oils suitable for the implementation of the invention, mention can particularly be made of:
hydrocarbon oils of animal origin,
hydrocarbon oils of plant origin, such as phytostearyl esters, such as phytostearyl oleate, phytostearyl isostearate and lauroyl/octyldodecyl/phytostearyl glutamate, for example sold under the name ELDEW PS203® by AJINOMOTO, triglycerides consisting of esters of fatty acids and glycerol for which the fatty acids can have chain lengths ranging from C₄ to C₂₄, with the latter able to be linear or branched, saturated or unsaturated; these oils are particularly heptanoic or octanoic oils, wheat germ, sunflower, grape seed, sesame, corn, apricot, castor, camelina, shea, avocado, olive, soybean, sweet almond, palm, rapeseed, cotton, hazelnut, macadamia, jojoba, alfalfa, poppy seed, pumpkin, squash, blackcurrant, evening primrose, millet, barley, quinoa, rye, safflower, candlenut, passiflora, musk rose oils; or caprylic/capric acid triglycerides such as those sold by Stearineries Dubois or those sold under the names Miglyol 810, 812 and 818® by Dynamit Nobel, refined plant perhydrosqualene sold under the name Fitoderm by Cognis,
synthetic esters such as oils of formula R₁COOR₂, wherein R₁ represents a linear or branched fatty acid residue including 1 to 40 carbon atoms, and R₂ represents a hydrocarbon-based chain, particularly a branched chain, including 1 to 40 carbon atoms provided that the sum of R₁ and R₂ is greater than or equal to 10. The esters can particularly be selected from esters, particularly fatty acid esters, such as for example cetostearyl octanoate, isopropyl alcohol and C8-C18, preferably C12-C16, fatty acid esters, such as isopropyl palmitate, ethyl palmitate, 2-ethylhexyl palmitate, isopropyl stearate or isostearate, isostearyl isostearate, octyl stearate, hydroxylated esters, for instance isostearyl lactate, octyl hydroxystearate, diisopropyl adipate, heptanoates, and particularly isostearyl heptanoate, alcohol or polyalcohol octanoates, decanoates or ricinoleates, such as propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethylhexanoate, and mixtures thereof, C₁₂-C₁₅ alkyl benzoates, hexyl laurate, neopentanoic acid esters, for instance isodecyl neopentanoate, isotridecyl neopentanoate, isostearyl neopentanoate, or octyldodecyl neopentanoate, isononanoic acid esters, such as isononyl isononanoate, isotridecyl isononanoate, octyl isononanoate, hydroxylated esters such as isostearyl lactate, diisostearyl malate;
polyol esters, and pentaerythritol esters, such as dipentaerythrityl tetrahydroxystearate/tetraisostearate,
esters of diol dimers and diacid dimers, such as Lusplan DD-DA5® and Lusplan DD-DA7®, sold by the NIPPON FINE CHEMICAL COMPANY and described in the application FR 03 02809,
fatty alcohols that are liquid at ambient temperature, with a branched and/or unsaturated carbon chain having 12 to 26 carbon atoms, preferably 16 to 22 carbon atoms, more preferably 18 to 20 carbon atoms, such as 2-octyldodecanol, isostearyl alcohol, oleic alcohol, 2-hexyldecanol, 2-butyloctanol, and 2-undecylpentadecanol,
higher fatty acids, such as oleic acid, linoleic acid, linolenic acid, and mixtures thereof,
diesters of C2-C16, preferably C8-C12, dicarboxylic acid and C1-04 monoalcohol, preferably of C3-C4 branched monoalcohol; preferably the diester of sebacic acid and isopropyl alcohol such as the diisopropyl sebacate sold under the name DUB DIS by STÉARINERIES DUBOIS,
the non-volatile silicone oils, for example non-volatile polydimethylsiloxanes (PDMS), polydimethylsiloxanes comprising alkyl or alkoxy groups which are pendant and/or at the end of the silicone chain, groups each having from 2 to 24 carbon atoms, phenylated silicones such as phenyl trimethicones, phenyl dimethicones, phenyltrimethylsiloxydiphenylsiloxanes, diphenyl dimethicones, diphenylmethyldiphenyltrisiloxanes, and (2-phenylethyl)trimethylsiloxysilicates, dimethicones or phenyltrimethicone of viscosity less than or equal to 100 Cst, and
mixtures thereof.

### Volatile Additional Polar Oils

For the purposes of this invention, the term “volatile oil” denotes an oil (or non-aqueous medium) liable to evaporate on contact with the skin in less than one hour, at ambient temperature and atmospheric pressure. The volatile oil is a volatile cosmetic oil, liquid at ambient temperature, particularly having a vapor pressure different to zero, at ambient temperature and atmospheric pressure, particularly having a vapor pressure ranging from 0.13 Pa to 40,000 Pa (10⁻³ at 300 mm Hg), in particular ranging from 1.3 Pa to 13,000 Pa (0.01 to 100 mm Hg), and preferentially ranging from 1.3 Pa to 1300 Pa (0.01 at 10 mm Hg).

It is possible to use, as volatile oils, volatile silicones, such as, for example, volatile linear or cyclic silicone oils, in particular those having a viscosity below or equal to 8 centistokes (8.10⁻⁶ m²/s), and having particularly from 2 to 10 silicon atoms, and in particular from 2 to 7 silicon atoms, these silicones optionally comprising alkyl or alkoxyl groups having from 1 to 10 carbon atoms. By way of volatile silicone oil that can be used in the invention, mention can be made particularly of dimethicones with a viscosity of 5 and 6 cSt, octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof. Volatile fluorinated oils such as nonafluoromethoxybutane or perfluoromethylcyclopentane and mixtures thereof can also be used. A mixture of the oils cited above can also be used.

According to an embodiment, the additional polar oil(s) is(are) non-volatile. Preferably, the additional polar oil(s) is(are) chosen from fatty alcohols that are liquid at ambient temperature with a branched and/or unsaturated carbon chain having 12 to 26 carbon atoms, preferably 16 to 22 carbon atoms, preferably 18 to 20 carbon atoms, and mixtures thereof.

Preferably, the composition according to the invention comprises at least one non-volatile additional polar oil chosen from fatty alcohols that are liquid at ambient temperature with a branched carbon chain having 12 to 26 carbon atoms, preferably 16 to 22 carbon atoms, preferably 18 to 20 carbon atoms, and at least one non-volatile additional polar oil chosen from fatty alcohols that are liquid at ambient temperature with an unsaturated carbon chain having 12 to 26 carbon atoms, preferably 16 to 22 carbon atoms, preferably 18 to 20 carbon atoms.

Preferably, the composition according to the invention comprises at least one non-volatile additional polar oil chosen from octyldodecanol, oleic alcohol and mixtures thereof.

Preferably, the composition according to the invention comprises isopropyl myristate or dicaprylyl carbonate, and at least one non-volatile additional polar oil chosen from octyldodecanol, oleic alcohol and mixtures thereof.

Preferably, isopropyl myristate and/or dicaprylyl carbonate are present at a content between 7% and 35% by weight, preferably between 8% and 30% by weight with respect to the total weight of the composition.

Preferably, the additional polar oil(s) chosen from fatty alcohols that are liquid at ambient temperature, with a branched and/or unsaturated carbon chain having 12 to 26 carbon atoms is(are) present in the composition at a concentration between 4% and 60% by weight, preferably between 10% and 55% by weight, preferably between 25% and 40% by weight with respect to the total weight of the composition.

Preferably, the composition according to the invention comprises:
from 7% to 35% by weight, preferably from 8% to 30% by weight with respect to the total composition weight, of isopropyl myristate or dicaprylyl carbonate;
from 0% to 45% by weight, preferably from 0.1% to 45% by weight, preferably from 5% to 41% by weight with respect to the total composition weight of octyldodecanol, and
from 0% to 15% by weight, preferably from 0.1% to 15% by weight, preferably from 2% to 11% by weight with respect to the total composition weight of oleic acid.

According to another embodiment, the composition according to the invention preferably comprises from 10% to 60% by weight of octyldodecanol, preferably from 10% to 45% by weight, preferably from 15% to 35% by weight with respect to the total weight of the composition.

### Additional Fatty Substances

The composition according to the invention can comprise additional fatty substances different from the polar oils cited above, and particularly one or more non-polar oils and/or one or more solid and/or pasty fats.

The term “non-polar oil”, for the purposes of this invention, denotes an oil wherein the solubility parameter δₐ at 25° C. as defined above is equal to 0 (J/cm³)^{1/2}.

Among the non-polar oils, mention can be made for example of:

### Non-Volatile Non-Polar Oils

Hydrocarbon oils of mineral or synthetic origin, such as for example: linear or branched hydrocarbons of mineral or synthetic origin, such as petroleum jelly, polydecenes, hydrogenated polyisobutene such as Parleam, squalane, and mixtures thereof, and in particular hydrogenated polyisobutene.

### Volatile Non-Polar Oils

Volatile hydrocarbon oils can be selected from hydrocarbon oils having from 8 to 16 carbon atoms, and particularly branched C₈-C₁₆ alkanes (also known as isoparaffins) such as isododecane (also known as 2,2,4,4,6-pentamethylheptane), isodecane, isohexadecane, and for example the oils sold under the trade names Isopars® or Permethyls®; linear alkanes, for example n-dodecane (C12) and n-tetradecane (C14) sold by Sasol respectively under the references PARAFOL 12-97 and PARAFOL 14-97, as well as the mixtures thereof, the undecane-tridecane mixture (Cetiol UT®), the mixtures of n-undecane (C11) and n-tridecane (C13) obtained in examples 1 and 2 of application WO2008/155059 of Cognis and mixtures thereof.

Preferably, the non-polar oil is selected from non-volatile non-polar oils, more preferably the non-polar oil is selected from hydrocarbon oils of mineral or synthetic origin, in particular linear or branched hydrocarbons, of mineral or synthetic origin. In a preferred embodiment, the non-polar oil is squalane or hemisqualane.

Preferably, the composition according to the invention comprises less than 10% by weight of non-polar oil with respect to the total weight of the composition, preferentially less than 6% by weight of non-polar oil, more preferably less than 5% by weight of non-polar oil with respect to the total weight of the composition, or the composition is free from non-polar oil.

The other fats which can be present in the composition according to the invention are particularly solid and/or pasty fats.

The term “solid fat” denotes any fatty substance in solid form at ambient temperature (25° C.) and at atmospheric pressure.

The term “pasty fat” denotes a lipophilic fat compound having a reversible solid/liquid change of state, having in the solid state, an anisotropic crystalline organization, and comprising at a temperature of 23° C. a liquid fraction and a solid fraction. In other words, the initial melting point of the pasty fat can be less than 23° C. The liquid fraction of the pasty fat measured at 23° C. can represent 9 to 97% by weight of the pasty fat. This liquid fraction at 23° C. preferably represents between 15 and 85%, more preferably between 40 and 85% by weight.

The melting point is equivalent to the temperature of the most endothermic peak observed in thermal analysis (DSC) as described in the standard ISO 11357-3; 1999. The melting point of a pasty fat can be measured using a differential scanning calorimeter (DSC), for example the calorimeter sold under the name “MDSC 2920” by TA Instruments.

According to a specific embodiment of the invention, the composition according to the invention comprises less than 3% by weight of solid and/or pasty fats, preferentially less than 2% by weight of solid and/or pasty fats, more preferably less than 1% by weight of solid and/or pasty fats with respect to the total weight of the composition. Preferably, the composition is free from solid and/or pasty fats.

The composition according to the invention also comprises at least 10% by weight, preferably at least 32% by weight with respect to the total weight of the composition of water-miscible organic solvent(s) chosen from polyols having 2 to 20 carbon atoms optionally in a mixture with at least one C₂-C₆ aliphatic monoalcohol.

Thus, the composition according to the invention comprises at least one polyol having 2 to 20 carbon atoms, said polyol optionally being present with at least one C₂-C₆ aliphatic monoalcohol.

### Polyol

The composition according to the invention comprises at least one polyol having 2 to 20 carbon atoms.

Said polyol is, preferably, chosen from polyols having 2 to 6 carbon atoms, such as glycerol, diglycerol, propyleneglycol, isoprene glycol, dipropyleneglycol, butylene glycol, hexylene glycol, 1,3-propanediol, pentylene glycol, polyethyleneglycols having 2 to 200 ethylene oxide units and mixtures thereof.

Preferably, the polyol having 2 to 20 carbon atoms is chosen from hexylene glycol, dipropylene glycol and mixtures thereof.

Preferably, the composition comprises from 10% to 65% by weight of polyol(s) having 2 to 20 carbon atoms, with respect to the total weight of the composition, more preferably from 20% to 60% by weight, even more preferably from 27 to 55% by weight.

### C₂-C₆ Monoalcohol

The composition according to the invention can comprise at least one C₂-C₆ aliphatic monoalcohol. Preferably, said aliphatic monoalochol comprises 2 to 4 carbon atoms.

The term “aliphatic monoalcohol” denotes any saturated, linear or branched alkane compound having a single hydroxyl (OH) function. The aliphatic monoalcohol(s) present in the compositions according to the invention can be selected from ethanol, propanol, butanol, isopropanol, isobutanol or mixtures thereof.

More specifically, ethanol will be selected.

Preferably, the composition according to the invention comprises from 0% to 15% by weight of at least one C₂-C₆ aliphatic monoalcohol (preferably ethanol), preferably from 0.1% to 15% by weight, preferably from 2% to 11% by weight of at least one C₂-C₆ aliphatic monoalcohol.

Preferably, the composition according to the invention comprises at least one polyol having 2 to 20 carbon atoms chosen from hexylene glycol, dipropylene glycol and mixtures thereof, and optionally at least ethanol.

Preferably, the composition according to the invention comprises at least two compounds chosen from categories (i) to (iv) hereinafter, on the condition that at least the two compounds belong to different categories:
(i) polyols having 2 to 20 carbon atoms,
(ii) C₂-C₆ aliphatic monoalcohols,
(iii) fatty alcohols that are liquid at ambient temperature with a branched carbon chain having 12 to 26 carbon atoms, such as 2-octyldodecanol, isostearyl alcohol, 2-hexyldecanol, 2-butyloctanol and 2-undecylpentadecanol, and
(iv) fatty alcohols that are liquid at ambient temperature with an unsaturated carbon chain having 12 to 26 carbon atoms, such as oleic alcohol.

Preferably, the composition according to the invention has a weight ratio between the oily phase and the hydrophilic phase between 0.1 and 4, preferably between 0.5 and 3.6, preferably between 0.5 and 2.6.

The oily phase according to the invention comprises the polar oil(s) according to the invention, and optionally the other lipophilic compounds. The hydrophilic phase according to the invention comprises the polyol having 2 to 20 carbon atoms, optionally the C₂-C₆ aliphatic monoalcohol, the hydrophilic active agent, and optionally water.

Preferably, the weight ratio between the oily phase and the hydrophilic phase is between 0.1 and 4, preferably between 0.5 and 2.6.

### Hydrophilic Active Agent

The composition comprises at least one hydrophilic active agent.

The term “hydrophilic active agent” denotes a water-soluble or water-dispersible active agent capable of forming hydrogen bonds.

By way of hydrophilic active agents, mention can be made for example of moisturizing agents; depigmenting agents, desquamating agents, anti-aging agents, matting agents; healing agents; antibacterial agents; and mixtures thereof.

Preferably, the composition according to the invention comprises from 0.1% to 20% by weight of at least one hydrophilic active agent, preferentially from 1% to 15% by weight, and preferentially from 2% to 12% by weight of at least one hydrophilic active agent with respect to the total weight of the composition.

In particular, the composition according to the invention can comprise at least one hydrophilic active agent selected from the C-glycoside derivatives of the following general formula (I):

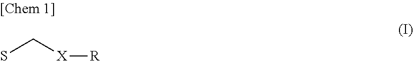

wherein:
   R designates a non-substituted C₁-C₄, particularly C₁-C₂, linear alkyl radial, in particular methyl;
   S represents a monosaccharide selected from D-glucose, D-xylose, N-acetyl-D-glucosamine or L-fucose, and in particular D-xylose;
   X represents a group selected from —CO—, —CH(OH)—, —CH(NH₂)—, and preferentially a —CH(OH)— group;
      as well the cosmetically acceptable salts thereof, solvates thereof such as hydrates and the optical isomers thereof.

By way of illustration and not limitation of the C-glycosides of formula (I) more specifically suitable for the invention, mention can particularly be made of the following compounds:
C-beta-D-xylopyranoside-n-propane-2-one;
C-alpha-D-xylopyranoside-n-propane-2-one;
C-beta-D-xylopyranoside-2-hydroxy-propane;
C-alpha-D-xylopyranoside-2-hydroxy-propane;
1-(C-beta-D-glucopyranosyl)-2-hydroxy-propane;
1-(C-alpha-D-glucopyranosyl)-2-hydroxy-propane;
1-(C-beta-D-glucopyranosyl)-2-amino-propane;
1-(C-alpha-D-glucopyranosyl)-2-amino-propane;
3′-(acetamido-C-beta-D-glucopyranosyl)-propane-2′-one;
3′-(acetamido-C-alpha-D-glucopyranosyl)-propane-2′-one;
1-(acetamido-C-beta-D-glucopyranosyl)-2-hydroxyl-propane;
1-(acetamido-C-beta-D-glucopyranosyl)-2-amino-propane;
as well the cosmetically acceptable salts thereof, solvates thereof such as hydrates and the optical isomers thereof.

Preferably, C-beta-D-xylopyranoside-2-hydroxy-propane or C-alpha-D-xylopyranoside-2-hydroxy-propane is used, and more preferably C-beta-D-xylopyranoside-2-hydroxy-propane. Preferably, a C-glycoside of formula (I) suitable for the invention can be advantageously C-beta-D-xylopyranoside-2-hydroxy-propane, of which the INCI name is HYDROXYPROPYL TETRAHYDROPYRANTRIOL particularly sold under the name MEXORYL SBB® or MEXORYL SON® by CHIMEX. Preferably, such a C-glycoside is present in concentrated form, i.e., in a hydrophilic dispersion concentrated to 70% with active substance; such a C-glycoside is marketed under the name MEXORYL SCS by NOVEAL. The salts of the C-glycosides of formula (I) suitable for the invention can comprise conventional physiologically acceptable salts of these compounds such as those formed from organic or inorganic acids. By way of example, mention can be made of the salts of mineral acids, such as sulfuric acid, hydrochloric acid, hydrobromic acids, hydroiodic acid, phosphoric acid and boric acid. Mention can also be made of organic salts, which can include one or more carboxylic, sulfonic, or phosphonic acid groups. They can consist of linear, branched or cyclic aliphatic acids or indeed aromatic acids. These acids can further include one or more heteroatoms selected from O and N, for example in the form of hydroxyl groups. Mention can particularly be made of propionic acid, acetic acid, terephthalic acid, citric acid, and tartaric acid.

The acceptable solvates for the compounds described above comprise conventional solvates such as those formed during the final preparation step of said compounds due to the presence of solvents. By way of example, mention can be made of solvates due to the presence of water or linear or branched alcohols such as ethanol or isopropanol.

The C-glycosides (I) are known from the document WO 02/051828.

According to an embodiment, the composition according to the invention comprises a C-glycoside in a quantity between 0.5% and 15% by weight of active substance (C-glycoside) with respect to the total weight of the composition, in particular between 1% and 10% by weight of active substance with respect to the total weight of the composition, more specifically between 2% and 5% by weight of active substance with respect to the total weight of the composition.

The compositions according to the invention can contain one or more of the usual adjuvants in the cosmetic and dermatological fields, gelling agents and/or lipophilic thickeners; moisturizing agents; emollients; lipophilic active agents; anti-free radical agents; sequestrants; fillers; antioxidants, such as pentaerythrityl tetra-di-t-butyl hydroxyhydrocinnamate; essential oils; perfumes; film-forming agents; soluble dyes; and mixtures thereof. The quantities of these different adjuvants are those conventionally used in the fields in question. In particular, these quantities vary according to the sought purpose and can for example range from 0.01% to 20%, and preferably from 0.1% to 10% by weight of the total weight of the composition.

Obviously, those skilled in the art will take care to choose the optional adjuvant(s) added to the composition according to the invention in such a way that the advantageous properties intrinsically associated with the composition according to the invention are not altered, or are not substantially altered, by the envisaged addition.

By way of gelling agents and/or lipophilic thickeners, mention can be made of esters of dextrin and fatty acids, in particular dextrin palmitates such as for example those sold under the name RHEOPEARL TL2-OR or RHEOPEARL KL2-OR by CHIBA FLOUR MILLING, and under the name RHEOPEARL KS by CHIBA FLOUR MILLING, and dextrin myristates, such as for example those sold under the name RHEOPEARL MKL2 by CHIBA FLOUR MILLING. Mention can further be made of tri-esters of fatty acids and mono or polyglycerol such as glyceryl tri-(hydroxystearate) (INCI name: TRIHYDROXYSTEARIN) such as for example that sold by ELEMENTIS under the name THIXCIN R or that which is sold by BYK ADDITIVES & INSTRUMENTS under the name RHEOCIN; modified clays such as hectorite and derivatives thereof, such as the products under the names Bentone.

Preferentially, the composition comprises less than 3% by weight of gelling agent and/or lipophilic thickener, preferably less than 2% by weight of gelling agent and/or lipophilic thickener with respect to the total weight of the composition, more preferably, less than 1% by weight of gelling agent and/or lipophilic thickener with respect to the total weight of the composition. In a specific embodiment, the composition is free from gelling agent and/or lipophilic thickener.

The composition according to the invention can further comprise additional active agents different from the hydrophilic active agents cited above.

The quantities of these different active agents are those conventionally used in the fields in question. In particular, these quantities vary according to the sought purpose and can for example range from 0.01% to 20%, and preferably from 0.1% to 10% by weight of the total weight of the composition.

Obviously, those skilled in the art will take care to choose the optional active agent(s) added to the composition according to the invention in such a way that the advantageous properties intrinsically associated with the composition according to the invention are not altered, or are not substantially altered, by the envisaged addition.

The composition can further comprise one or more UV filters, in particular organic filters, particularly liposoluble organic filters.

According to a particular embodiment, the composition according to the invention comprises less than 2% by weight of filler with respect to the total weight of the composition, more preferably, the composition according to the invention is free from filler. The term “filler” should be understood to denote inorganic or synthetic, colorless or white particles of any shape, insoluble in the medium of the composition regardless of the temperature at which the composition is manufactured.

Preferably, the transparent single-phase composition according to the invention consists of:
from 7% to 35% by weight, preferably from 8% to 30% by weight with respect to the total composition weight, of isopropyl myristate or dicaprylyl carbonate;
from 0% to 45% by weight, preferably from 0.1% to 45% by weight, preferably from 5% to 41% by weight with respect to the total composition weight of octyldodecanol,
from 0% to 15% by weight, preferably from 0.1% to 15% by weight, preferably from 2% to 11% by weight with respect to the total composition weight of oleic acid,
from 10% to 65% by weight of polyol(s) having 2 to 20 carbon atoms, with respect to the total weight of the composition, more preferably from 20% to 60% by weight, even more preferably from 27 to 55% by weight, said polyol being chosen from hexylene glycol, dipropylene glycol and mixtures thereof,
from 0% to 15% by weight of at least one C₂-C₆ aliphatic monoalcohol, preferably ethanol, preferably from 0.1% to 15% by weight, preferably from 2% to 11% by weight,
from 0.1% to 20% by weight of hydrophilic active agent, preferably from 1% to 15% by weight, and preferably from 2% to 12% by weight with respect to the total weight of the composition, preferably from 0.5% to 15% by weight, preferably from 1% to 10% by weight, and preferably from 2% to 5% by weight with respect to the total weight of the C-glycoside composition chosen from C-beta-D-xylopyranoside-2-hydroxy-propane and C-alpha-D-xylopyranoside-2-hydroxy-propane, preferably C-beta-D-xylopyranoside-2-hydroxy-propane, and
the composition comprising less than 8% by weight of water with respect to the total weight of the composition, preferably less than 4% by weight of water, preferably less than 3% by water, preferably less than 2% by weight of water, preferably less than 1.5% by weight of water, preferably less than 1.1% by weight of water.

According to another embodiment, the composition according of the invention preferably consists of:
from 30% to 70% by weight, preferably from 40% to 65% by weight, more preferably from 45% to 60% by weight with respect to the total weight of the composition of octyldodecanol;
from 0% to 15% by weight, preferably from 0.1% to 15% by weight, preferably from 2% to 11% by weight with respect to the total composition weight of oleic acid,
from 10% to 65% by weight of polyol(s) having 2 to 20 carbon atoms, with respect to the total weight of the composition, more preferably from 20% to 60% by weight, even more preferably from 27 to 55% by weight, said polyol being chosen from hexylene glycol, dipropylene glycol and mixtures thereof,
from 0% to 15% by weight of at least one C₂-C₆ aliphatic monoalcohol, preferably ethanol, preferably from 0.1% to 15% by weight, preferably from 2% to 11% by weight,
from 0.1% to 20% by weight of hydrophilic active agent, preferably from 1% to 15% by weight, and preferably from 2% to 12% by weight with respect to the total weight of the composition, preferably from 0.5% to 15% by weight, preferably from 1% to 10% by weight, and preferably from 2% to 5% by weight with respect to the total weight of the C-glycoside composition chosen from C-beta-D-xylopyranoside-2-hydroxy-propane and C-alpha-D-xylopyranoside-2-hydroxy-propane, preferably C-beta-D-xylopyranoside-2-hydroxy-propane, and
the composition comprising less than 8% by weight of water with respect to the total weight of the composition, preferably less than 4% by weight of water, preferably less than 3% by water, preferably less than 2% by weight of water, preferably less than 1.5% by weight of water, preferably less than 1.1% by weight of water.

The invention also relates to the cosmetic use of a composition according to the invention for the care of keratin materials, in particular for body and/or facial skin care.

The present invention further relates to a non-therapeutic cosmetic care method of keratin materials such as the skin, in particular body and/or facial skin, wherein a composition according to the invention is applied on said keratin materials.

The composition according to the invention can be obtained conventionally by those skilled in the art.

The following examples make it possible to understand the invention better, without being in any way limitative. The raw materials are referred to with the chemical or INCI name thereof. The quantities are indicated by weight of raw materials with respect to the total weight of the composition, unless specified otherwise.

### EXAMPLE 1: PREPARATIONS OF COMPOSITIONS ACCORDING TO THE INVENTION

Compositions F1 to F6 were prepared according to the following method:
a) Produce a pre-dispersion of the hydrophilic active agent in the mixture of glycols and monoalcohol under magnetic stirring or a deflocculator until a perfectly homogeneous and clear mixture is obtained; then
b) Add the oily phase until a perfectly homogeneous and clear mixture is obtained.

TABLE 1

| | F1 | F2 | F3 | F4 | F5 | F6 |
|---|---|---|---|---|---|---|
| Isopropyl myristate | — | — | 27.70 | — | — | — |
| (ISOPROPYLMYRISTATE BASF) | | | | | | |
| Castor oil | — | — | — | — | — | 27.7 |
| Octyldodecanol | Qs 100 | Qs 100 | Qs 100 | Qs 100 | Qs 100 | Qs 100 |
| (Eutanol G ®, BASF) | | | | | | |
| Dicaprylyl carbonate | 24 | 30 | — | 27.70 | — | — |
| (Cetiol CC, Cognis) | | | | | | |
| Hexylene glycol | 14 | 28 | 14 | 14 | 14 | 14 |
| Dipropylene glycol | 15.50 | — | 15.50 | 15.50 | 15.50 | 15.50 |
| Ethanol | 5 | 10 | 4 | 4 | 4 | 4 |
| Niacinamide | 3 | | — | — | — | — |
| Perfume | Qs | — | — | — | — | — |
| Capryloyl salicylic acid | 0.30 | — | — | — | — | — |
| Oleic alcohol | 10 | 10 | 10 | 10 | 10 | 10 |
| (HD OCENOL 80/85 V/MB ®, | | | | | | |
| BASF) | | | | | | |
| Hydroxypropyl tetrahydropyrantriol | 4.5 | 4.5 | 4.3 | 4.3 | 4.3 | 4.3 |
| (MEXORYL SCS , NOVEAL | (= | (= | (= | (= | (= | (= |
| (70% AS* in 22% water and 8% | 3.15% | 3.15% | 3.01% | 3.01% | 3.01% | 3.01% |
| propylene glycol)) | AS) | AS) | AS) | AS) | AS) | AS) |

*AS: active substance

### For F1:

The oily phase represents 57.7%, and the hydrophilic phase represents 42% (to which the 0.3% capryloyl salicylic acid is added), i.e., a fatty phase/hydrophilic phase ratio equal to 1.37.

### For F2:

The oily phase represents 57.5%, and the hydrophilic phase represents 42.5%, i.e., a fatty phase/hydrophilic phase ratio equal to 1.35.

### For F3:

The oily phase represents 62.2%, and the hydrophilic phase represents 37.8%, i.e., a fatty phase/hydrophilic phase ratio equal to 1.65.

### For F4:

The oily phase represents 62.2%, and the hydrophilic phase represents 37.8%, i.e., a fatty phase/hydrophilic phase ratio equal to 1.65.

Formulas F3 to F6 were tested for the penetration time and oily finish thereof with the following test:

Protocol: for n =from 12 to 15 candidates, application of 0.20 ml on an area of the hand equivalent to 5 cm², the penetration time is counted in seconds until the area seems dry, and the oily finish is evaluated on a scale going from 1 (not oily) to 5 (very oily). The results are gathered in the following tables:

| | Penetration time | Oily finish |
|---|---|---|
| F4 | 14.8 | 2.3 |
| F5 | 17.3 | 2.8 |
| F6 | 21.0 | 3.5 |
| F3 | 9.9 | 1.5 |
| F6 | 14.0 | 2.5 |

After application, formulas F3 to F5 according to the invention present a perception of less greasy finish and a perception of faster penetration speed as compared to the composition F6.

### EXAMPLE 2: PREPARATION OF COMPOSITIONS ACCORDING TO THE INVENTION (SINGLE-PHASE) AND COMPARATIVE COMPOSITIONS (NON-SINGLE-PHASE)

The following compositions were prepared as in example 1.

The evaluation of the appearance thereof under stirring, after 10 minutes and after 24h, was performed according to the following score:
Score of 1=clear and single-phase
Score of 2=opalescent
Score of 3=turbid

Score of 4=opaque
Score of 5=bi-phase.

The results are in the table below (the quantities are by weight with respect to the total composition weight, to which 4.3% Hydroxypropyl tetrahydropyrantriol active agent (Mexoryl SCS from Noveal) should be added. The compositions according to the invention are marked with an asterisk, the others are comparative:

TABLE 2

| | | | | | | | | After | |
|---|---|---|---|---|---|---|---|---|---|
| | Dipropylene | Hexylene | | | Oleic | Isopropyl | Under | 10 | After |
| Experiment | glycol | glycol | Ethanol | Octyldodecanol | alcohol | myristate | stirring | min | 24 h |
| 47 | 0.00% | 10.00% | 0.00% | 80.70% | 5.00% | 0.00% | 3 | 5 | 5 |
| 60 | 30.00% | 0.00% | 0.00% | 60.70% | 5.00% | 0.00% | 2 | 2 | 2 |
| 67* | 8.23% | 33.23% | 7.78% | 35.10% | 2.78% | 8.58% | 1 | 1 | 1 |
| 68 | 13.23% | 8.23% | 2.78% | 60.10% | 2.78% | 8.58% | 2 | 2 | 2 |
| 28* | 0.00% | 50.00% | 0.00% | 25.70% | 5.00% | 15.00% | 1 | 1 | 1 |
| 32* | 0.00% | 50.00% | 5.00% | 15.70% | 10.00% | 15.00% | 1 | 1 | 1 |
| 40* | 50.00% | 0.00% | 5.00% | 25.70% | 0.00% | 15.00% | 1 | 1 | 1 |
| 48 | 0.00% | 10.00% | 0.00% | 70.70% | 0.00% | 15.00% | 3 | 5 | 5 |
| 49 | 10.00% | 0.00% | 0.00% | 60.70% | 10.00% | 15.00% | 2 | 5 | 5 |
| 55* | 0.00% | 30.00% | 10.00% | 40.70% | 0.00% | 15.00% | 1 | 1 | 1 |
| 56 | 0.00% | 30.00% | 0.00% | 40.70% | 10.00% | 15.00% | 3 | 2 | 2 |
| 58 | 30.00% | 0.00% | 0.00% | 50.70% | 0.00% | 15.00% | 2 | 5 | 5 |
| 62* | 15.00% | 15.00% | 10.00% | 40.70% | 0.00% | 15.00% | 1 | 1 | 1 |
| 59* | 33.14% | 0.00% | 10.00% | 25.42% | 10.00% | 17.14% | 1 | 1 | 1 |
| 66 | 8.23% | 13.23% | 7.78% | 35.10% | 7.78% | 23.58% | 2 | 2 | 2 |
| 69* | 33.23% | 8.23% | 5.63% | 17.25% | 7.78% | 23.58% | 1 | 1 | 1 |
| 17* | 50.00% | 0.00% | 10.00% | 0.00% | 10.00% | 25.70% | 1 | 1 | 1 |
| 45* | 25.00% | 25.00% | 10.00% | 0.00% | 10.00% | 25.70% | 1 | 1 | 1 |
| 6* | 0.00% | 50.00% | 0.00% | 5.70% | 10.00% | 30.00% | 1 | 1 | 1 |
| 7* | 0.00% | 50.00% | 10.00% | 5.70% | 0.00% | 30.00% | 1 | 1 | 1 |
| 12 | 50.00% | 0.00% | 0.00% | 15.70% | 0.00% | 30.00% | 3 | 5 | 5 |
| 14 | 50.00% | 0.00% | 10.00% | 5.70% | 0.00% | 30.00% | 2 | 2 | 2 |
| 15* | 0.00% | 45.70% | 10.00% | 0.00% | 10.00% | 30.00% | 1 | 1 | 1 |
| 16* | 50.00% | 0.00% | 5.70% | 0.00% | 10.00% | 30.00% | 1 | 1 | 1 |
| 22 | 10.00% | 0.00% | 0.00% | 55.70% | 0.00% | 30.00% | 3 | 5 | 5 |
| 23 | 0.00% | 10.00% | 0.00% | 45.70% | 10.00% | 30.00% | 3 | 5 | 5 |
| 24 | 0.00% | 10.00% | 10.00% | 45.70% | 0.00% | 30.00% | 3 | 5 | 5 |
| 25 | 10.00% | 0.00% | 10.00% | 35.70% | 10.00% | 30.00% | 2 | 5 | 5 |
| 35* | 25.00% | 25.00% | 0.00% | 15.70% | 0.00% | 30.00% | 1 | 1 | 1 |
| 36 | 0.00% | 30.00% | 0.00% | 35.70% | 0.00% | 30.00% | 3 | 5 | 5 |
| 37* | 25.00% | 25.00% | 0.00% | 5.70% | 10.00% | 30.00% | 1 | 1 | 1 |
| 38* | 25.00% | 25.00% | 10.00% | 5.70% | 0.00% | 30.00% | 1 | 1 | 1 |
| 43 | 30.00% | 0.00% | 0.00% | 25.70% | 10.00% | 30.00% | 2 | 5 | 5 |
| 44* | 30.00% | 0.00% | 10.00% | 25.70% | 0.00% | 30.00% | 1 | 1 | 1 |
| 46* | 0.00% | 27.85% | 10.00% | 17.85% | 10.00% | 30.00% | 1 | 1 | 1 |
| 53 | 10.00% | 0.00% | 5.00% | 45.70% | 5.00% | 30.00% | 2 | 2 | 2 |
| 54 | 0.00% | 30.00% | 5.00% | 30.70% | 0.00% | 30.00% | 2 | 2 | 2 |
| 64 | 15.00% | 15.00% | 0.00% | 30.70% | 5.00% | 30.00% | 3 | 5 | 5 |
| 65* | 16.57% | 16.57% | 10.00% | 17.42% | 5.14% | 30.00% | 1 | 1 | 1 |

TABLE 3

| | | | | | | | | After | |
|---|---|---|---|---|---|---|---|---|---|
| | Dipropylene | Hexylene | | Oleic | Dicaprylyl | | Under | 10 | After |
| Experiment | glycol | glycol | Octyldodecanol | alcohol | carbonate | Ethanol | stirring | min | 24 h |
| 60 | 30.00% | 0.00% | 60.70% | 5.00% | 0.00% | 0.00% | 2 | 2 | 3 |
| 63 | 15.00% | 15.00% | 50.70% | 10.00% | 0.00% | 5.00% | 2 | 5 | 5 |
| 26* | 25.00% | 25.00% | 45.70% | 0.00% | 0.00% | 0.00% | 1 | 1 | 1 |
| 67* | 8.23% | 33.23% | 35.10% | 2.78% | 8.58% | 7.78% | 1 | 1 | 1 |
| 68 | 13.23% | 8.23% | 60.10% | 2.78% | 8.58% | 2.78% | 2 | 5 | 5 |
| 28* | 0.00% | 50.00% | 25.70% | 5.00% | 15.00% | 0.00% | 1 | 1 | 1 |
| 32* | 0.00% | 50.00% | 15.70% | 10.00% | 15.00% | 5.00% | 1 | 1 | 1 |
| 40* | 50.00% | 0.00% | 25.70% | 0.00% | 15.00% | 5.00% | 1 | 1 | 1 |
| 48 | 0.00% | 10.00% | 70.70% | 0.00% | 15.00% | 0.00% | 2 | 5 | 5 |
| 49 | 10.00% | 0.00% | 60.70% | 10.00% | 15.00% | 0.00% | 2 | 5 | 5 |
| 56 | 0.00% | 30.00% | 40.70% | 10.00% | 15.00% | 0.00% | 2 | 2 | 3 |
| 58 | 30.00% | 0.00% | 50.70% | 0.00% | 15.00% | 0.00% | 2 | 2 | 4 |
| 62* | 15.00% | 15.00% | 40.70% | 0.00% | 15.00% | 10.00% | 1 | 1 | 1 |
| 59* | 33.14% | 0.00% | 25.42% | 10.00% | 17.14% | 10.00% | 1 | 1 | 1 |
| 66 | 8.23% | 13.23% | 35.10% | 7.78% | 23.58% | 7.78% | 2 | 5 | 5 |
| 69* | 33.23% | 8.23% | 17.25% | 7.78% | 23.58% | 5.63% | 1 | 1 | 1 |
| 17* | 50.00% | 0.00% | 0.00% | 10.00% | 25.70% | 10.00% | 1 | 1 | 1 |
| 45* | 25.00% | 25.00% | 0.00% | 10.00% | 25.70% | 10.00% | 1 | 1 | 1 |
| 7* | 0.00% | 50.00% | 5.70% | 0.00% | 30.00% | 10.00% | 1 | 1 | 1 |
| 12 | 50.00% | 0.00% | 15.70% | 0.00% | 30.00% | 0.00% | 3 | 5 | 5 |
| 13 | 50.00% | 0.00% | 5.70% | 10.00% | 30.00% | 0.00% | 2 | 2 | 5 |
| 14 | 50.00% | 0.00% | 5.70% | 0.00% | 30.00% | 10.00% | 3 | 5 | 5 |
| 15 | 0.00% | 45.70% | 0.00% | 10.00% | 30.00% | 10.00% | 5 | 5 | 5 |
| 16* | 50.00% | 0.00% | 0.00% | 10.00% | 30.00% | 5.70% | 1 | 1 | 1 |
| 22 | 10.00% | 0.00% | 55.70% | 0.00% | 30.00% | 0.00% | 3 | 2 | 5 |
| 23 | 0.00% | 10.00% | 45.70% | 10.00% | 30.00% | 0.00% | 3 | 2 | 5 |
| 35* | 25.00% | 25.00% | 15.70% | 0.00% | 30.00% | 0.00% | 1 | 1 | 1 |
| 36 | 0.00% | 30.00% | 35.70% | 0.00% | 30.00% | 0.00% | 2 | 3 | 2 |
| 37* | 25.00% | 25.00% | 5.70% | 10.00% | 30.00% | 0.00% | 1 | 1 | 1 |
| 38* | 25.00% | 25.00% | 5.70% | 0.00% | 30.00% | 10.00% | 1 | 1 | 1 |
| 43 | 30.00% | 0.00% | 25.70% | 10.00% | 30.00% | 0.00% | 2 | 2 | 4 |
| 44 | 30.00% | 0.00% | 25.70% | 0.00% | 30.00% | 10.00% | 3 | 3 | 2 |
| 46* | 0.00% | 27.85% | 17.85% | 10.00% | 30.00% | 10.00% | 1 | 1 | 1 |
| 53 | 10.00% | 0.00% | 45.70% | 5.00% | 30.00% | 5.00% | 2 | 3 | 5 |
| 54 | 0.00% | 30.00% | 30.70% | 0.00% | 30.00% | 5.00% | 3 | 3 | 3 |
| 64 | 15.00% | 15.00% | 30.70% | 5.00% | 30.00% | 0.00% | 2 | 2 | 5 |
| 65* | 16.57% | 16.57% | 17.42% | 5.14% | 30.00% | 10.00% | 1 | 1 | 1 |

In conclusion, only the compositions according to the invention are single-phase at all the times observed.

1. A transparent single-phase cosmetic composition comprising:
from 0.1% to 89% by weight of at least one polar oil with respect to the total weight of the composition, said polar oil comprising at least one compound selected from the group consisting of isopropyl myristate, di-alkyl carbonates, the 2 alkyl chains optionally being identical or different, octyldodecanol, and mixtures thereof;
at least 10% by weight with respect to the total weight of the composition of water-miscible organic solvent(s) chosen from polyols having 2 to 20 carbon atoms optionally in a mixture with at least one C₂-C₆ aliphatic monoalcohol; and
at least one hydrophilic active agent,
said composition comprising less than 8% by weight of water with respect to the total weight of the composition.

2. The composition according to claim 1, wherein it comprises a total quantity of polar oil(s) ranging from 35% to 80% by weight with respect to the total weight of the composition.

3. The composition according to claim 1, wherein the di-alkyl carbonate is dicaprylyl carbonate.

4. The composition according to claim 1, which comprises at least isopropyl myristate or dicaprylyl carbonate, and at least one non-volatile additional polar oil, and/or at least 32% by weight with respect to the total weight of the composition of water-miscible organic solvent(s) chosen from polyols having 2 to 20 carbon atoms optionally in a mixture with at least one C₂-C₆ aliphatic monoalcohol.

5. The composition according to claim 1, which comprises at least isopropyl myristate or dicaprylyl carbonate, and wherein isopropyl myristate and/or dicaprylyl carbonate are present at a content between 7% and 35% by weight with respect to the total weight of the composition.

6. The composition according to claim 1, which comprises:
from 7% to 35% by weight with respect to the total composition weight, of isopropyl myristate or dicaprylyl carbonate;
from 0% to 45% by weight with respect to the total composition weight of octyldodecanol, and
from 0% to 15% by weight with respect to the total composition weight of oleic acid.

7. The composition according to claim 1, which comprises less than 4% by weight of water with respect to the total weight of the composition.

8. The composition according to claim 1, which comprises at least one polyol having 2 to 20 carbon atoms selected from the group consisting of glycerol, diglycerol, propyleneglycol, isoprene glycol, dipropyleneglycol, butylene glycol, hexylene glycol, 1,3-propanediol, pentylene glycol, polyethyleneglycols having 2 to 200 ethylene oxide units and mixtures thereof, and optionally at least ethanol.

9. The composition according to claim 1, which comprises from 10% to 65% by weight of polyol(s) having 2 to 20 carbon atoms, with respect to the total weight of the composition.

10. The composition according to claim 1, which comprises from 0% to 15% by weight of at least one C2-C6 aliphatic monoalcohol.

11. The composition according to claim 1, which has a weight ratio between the oily phase and the hydrophilic phase between 0.1 and 4.

12. The composition according to claim 1, wherein the hydrophilic active agent is selected from C-glycoside derivatives of the following general formula (I): 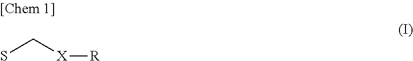
wherein:
R designates a non-substituted C₁-C₄linear alkyl radical;
S represents a monosaccharide selected from the group consisting of D-glucose, D-xylose, N-acetyl-D-glucosamine or L-fucose;
X represents a group selected from the group consisting of —O—, —CH(OH)—, —CH(NH₂);
as well at the cosmetically acceptable salts thereof, solvates thereof such as hydrates and the optical isomers thereof.

13. The composition according to claim 1, which comprises from 0.1% to 20% by weight of hydrophilic active agent with respect to the total weight of the composition.

14. The composition according to claim 1, which consists of:
from 7% to 35% by weight; with respect to the total composition weight, of isopropyl myristate or dicaprylyl carbonate;
from 0% to 45% by weight with respect to the total composition weight of octyldodecanol,
from 0% to 15% by weight with respect to the total composition weight of oleic acid,
from 10% to 65% by weight of polyol(s) having 2 to 20 carbon atoms, with respect to the total weight of the composition, said polyol being selected from the group consisting of chosen from hexylene glycol, dipropylene glycol and mixtures thereof,
from 0% to 15% by weight of at least one C₂-C₆ aliphatic monoalcohol,
from 0.1% to 20% by weight of hydrophilic active agent with respect to the total weight of the composition, and
the composition comprising less than 8% by weight of water with respect to the total weight of the composition.

15. A cosmetic treatment method of keratin materials, comprising the application, on said keratin materials, of a composition according to claim 1.

16. A cosmetic treatment method according to claim 15, for body and/or facial skin care.

17. The composition according to claim 2 wherein the di-alkyl carbonate is dicaprylyl carbonate.

18. The composition according to claim 2 which comprises at least isopropyl myristate or dicaprylyl carbonate, and at least one non-volatile additional polar oil and/or at least 32% by weight with respect to the total weight of the composition of water-miscible organic solvent(s) chosen from polyols having 2 to 20 carbon atoms optionally in a mixture with at least one C₂-C₆ aliphatic monoalcohol.

19. The composition according to claim 3 which comprises at least isopropyl myristate or dicaprylyl carbonate, and at least one non-volatile additional polar oil and/or at least 32% by weight with respect to the total weight of the composition of water-miscible organic solvent(s) chosen from polyols having 2 to 20 carbon atoms optionally in a mixture with at least one C₂-C₆ aliphatic monoalcohol.

20. The composition according to claim 2, which comprises at least isopropyl myristate or dicaprylyl carbonate, and wherein isopropyl myristate and/or dicaprylyl carbonate are present at a content between 7% and 35% by weight with respect to the total weight of the composition.
